SEQUENCE LISTING (3 sequences)

SEQ ID NO: 1 - protein, 4 aa, Artificial Sequence
  synthetic peptide
SDKP

SEQ ID NO: 2 - protein, 5 aa, Artificial Sequence
  synthetic peptide
HEMGH

SEQ ID NO: 3 - protein, 5 aa, Artificial Sequence
  synthetic peptide
KEMGK

(19) United States
(12) Patent Application Publication
(10) Pub. No.: US 2012/0088730 A1
(43) Pub. Date: Apr. 12, 2012

(54) USE OF ANGIOTENSIN CONVERTING ENZYME (ACE) DOMAIN SPECIFIC INHIBITORS TO INHIBIT OR ENHANCE CYTOKINE PRODUCTION AND ALTER IMMUNE RESPONSE

(75) Inventors: Kenneth E. Bernstein, Los Angeles, CA (US); Chentao Lin, Los Angeles, CA (US)

(73) Assignee: CEDARS-SINAI MEDICAL CENTER, Los Angeles, CA (US)

(21) Appl. No.: 13/375,301

(22) PCT Filed: Jun. 2, 2010
(86) PCT No.: PCT/US2010/037100

§ 371 Date: Nov. 30, 2011

Related U.S. Application Data
(60) Provisional application No. 61/183,499, filed on Jun. 2, 2009.

(51) Int. Cl. A61K 31/662; A61P 29/00; A61K 38/07; A61K 31/675; C12Q 1/37

(52) U.S. Cl. 514/16.3; 514/119; 514/80; 435/24

(57) ABSTRACT

The present invention relates to the discovery that the two ACE domains can have very different effects on cytokine expression. In one embodiment, the present invention provides a method of regulating and/or treating a condition in a subject by administering a therapeutically effective dosage of site specific ACE inhibitor. In another embodiment, the condition is inflammation and/or immune response.

### GOVERNMENT RIGHTS

The U.S. Government has a paid-up license in this invention and the right in limited circumstances to require the patent owner to license others on reasonable terms as provided for by the terms of Grant Nos. RO1 DK051445-12 and RO1 DK039777-22 awarded by the National Institutes of Health.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The classical renin-angiotensin system (RAS) is an enzymatic cascade producing the octapeptide angiotensin II from angiotensinogen. This occurs in two consecutive steps catalyzed by renin and ACE. While renin is very precise in its substrate specificity, ACE is more promiscuous. Angiotensin I and bradykinin are well known physiological substrates, but ACE also cleaves substance P, AcSDKP, β-endorphins, and several other peptides. Perhaps because of this variety of substrates, ACE affects many normal physiologic processes including blood pressure, hematopoiesis and fertility. ACE also plays a role in several pathological processes such as atherosclerosis and diabetic nephropathy.

ACE is a 170 kDa protein (a dipeptidyl carboxypeptidase) that is abundantly expressed by many tissues, including vascular endothelium, renal proximal tubule epithelium, and duodenal epithelium. Typically, it is an ectoenzyme bound to the outside of the cell membrane by a carboxyl terminal hydrophobic tail. While most ACE activity is incorporated in tissues such as the lung and the kidney, enzymatic cleavage of tissue-bound ACE results in a circulating form of the enzyme within plasma. ACE production by vascular endothelium generates angiotensin II in close proximity to vascular smooth muscle, a critical target organ for this vasoconstrictor. The ACE isozyme produced by endothelium and all other somatic tissues is called somatic ACE. Though a single polypeptide chain, somatic ACE is organized into two extracellular homologous domains, often termed the N- and C-terminal domains. Each of these domains contains the consensus amino acid sequence HEMGH, which binds zinc and is critical for the enzymatic activity of this metallopeptidase. Thus, while a single polypeptide, somatic ACE is composed of two catalytic domains, each of which binds zinc.

Generally, the inhibitors commonly used in clinical practice inhibit both cataytic domains of ACE. Few ACE inhibitors are used that specifically inhibit either N- or C-terminal ACE activity but not both. Thus, the discovery that the two catalytic domains of ACE have very different effects on cytokine expression could have enormous implications, including the basis for development and testing of additional site specific ACE inhibitors.

### SUMMARY OF THE INVENTION

Various embodiments include a method of regulating an inflammatory condition in a subject, comprising obtaining a sample from the subject, assaying the sample to determine the presence of a cytokine expression profile associated with an angiotensin converting enzyme (ACE) site specific catalytic domain, and regulating the inflammatory condition by administering to the subject a therapeutically effective amount of an inhibitor specific to the ACE site specific catalytic domain. In another embodiment, the ACE site specific catalytic domain is an ACE N-terminal site domain. In another embodiment, the ACE site specific catalytic domain is an ACE C-terminal site domain. In another embodiment, the cytokine expression profile comprises an TNF-α, IL-12, and/or IL-6 overexpression. In another embodiment, the TNF-α, IL-12, and/or IL-6 overexpression is associated with inactivation of an ACE N-terminal site domain. In another embodiment, the cytokine expression profile is associated with a MAP kinase expression profile. In another embodiment, the MAP kinase expression profile comprises phosphorylated JNK and/or Erk levels. In another embodiment, the cytokine expression profile is produced by interferon-γ (IFN-γ) and/or lipopolysacharide (LPS) stimulation. In another embodiment, the inhibitor specific to the ACE site specific catalytic domain comprises a compound the formula:

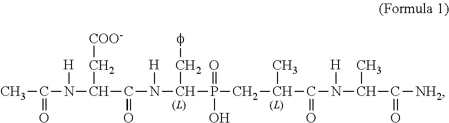

or a pharmaceutical equivalent, derivative, analog and/or salt thereof. In another embodiment, the inhibitor specific to the ACE site specific catalytic domain comprises a compound the formula:

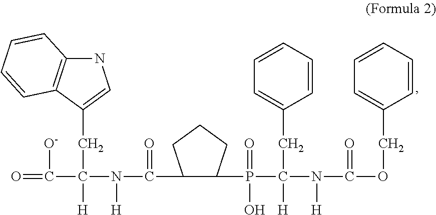

or a pharmaceutical equivalent, derivative, analog and/or salt thereof, and/or ketomethylene inhibitors. In another embodiment, the subject is a human. In another embodiment, the subject is a rodent. In another embodiment, the condition is regulated by AcSDKP. In another embodiment, the condition is regulated by angiotensin I, bradykinin, substance P and/or β-endorphins.

Other embodiments include a method of treating a disease and/or condition associated with angiotensin converting enzyme (ACE) in a subject, comprising providing a site specific ACE inhibitor, and treating the condition by administering a therapeutically effective dosage of the site specific ACE inhibitor to the subject. In another embodiment, the subject is a human. In another embodiment, the subject is a rodent. In another embodiment, the disease and/or condition associated with ACE is cancer. In another embodiment, the disease and/or condition associated with ACE is melanoma. In another embodiment, the disease and/or condition associated with ACE is blood pressure, hematopoiesis, fertility, atherosclerosis, diabetes and/or kidney disease. In another embodiment, the site specific ACE inhibitor comprises an ACE N-terminal domain inhibitor. In another embodiment, the site specific ACE inhibitor comprises an ACE C-terminal domain inhibitor.

Other embodiments include a method of treating cancer in a subject, comprising providing a composition comprising an agent that substantially inactivates the N-terminal catalytic domain of angiotensin-converting enzyme (ACE), and administering a therapeutically effective amount of the composition to the subject. In another embodiment, the subject is a rodent. In another embodiment, the subject is a human. In another embodiment, the cancer is melanoma.

Various embodiments include a method of developing and/or screening for an angiotensin converting enzyme (ACE) inhibitor, comprising determining the presence of an ACE domain that has an effective catalytic action, and developing an ACE inhibitor that specifically catalyses the ACE domain.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts examples of ACE structures in accordance with an embodiment of the present invention. Specifically, an example of a structure of somatic ACE and testis ACE are provided.

FIG. 2 depicts, in accordance with an embodiment of the present invention, various examples of ACE structures. The figure depicts a wild type ACE, an ACE with inactivated N-terminal catalytic site, and an ACE with inactivated C-terminal site. HEMGH is described herein as SEQ. ID. NO.: 2. KEMGK is described herein as SEQ. ID. NO.: 3.

FIG. 3 depicts, in accordance with an embodiment of the present invention, a sample of the Western blot data from the N-KO mice, establishing that the introduction of the point mutations has no effect on either the tissue pattern, the molecular size or the levels of ACE expression.

FIG. 4 depicts, in accordance with an embodiment of the present invention, systolic blood pressure, where there is no difference in blood pressure between N-KO, C-KO and wild type mice.

FIG. 5 depicts, in accordance with an embodiment of the present invention, TNF-α after LPS, where there was a marked difference in cytokine expression profile. Macrophages derived from N-KO mice producing far more TNF-α than either C-KO or WT mice.

FIG. 6 depicts, in accordance with an embodiment of the present invention, IL12p40 after LPS, where there was a marked difference in cytokine expression profile. Macrophages derived from N-KO mice producing far more IL-12p40 than either C-KO or WT mice.

FIG. 7 depicts, in accordance with an embodiment of the present invention, Western blot analysis of LPS stimulated p-JNK (minutes).

FIG. 8 depicts, in accordance with an embodiment of the present invention, Western blot analysis of LPS stimulated p-Erk ½.

FIG. 9 depicts, in accordance with an embodiment of the present invention, ACE7 (ACE N-terminus KO) are resistant to melanoma B16-F10, with (a) depicting F2 mice and (b) depicting F7 mice.

FIG. 10 depicts, in accordance with an embodiment of the present invention, CpG stimulated TNF expression in ACE7 (or ACE N-terminus KO) is higher than wild type and ACE13 (or ACE C-terminus KO).

FIG. 11 depicts, in accordance with an embodiment of the present invention, ACE inhibitor lisinopril can lower cytokine expression, but still cannot eliminate the difference between ACE7 (N-terminus KO) and wild type.

FIG. 12 depicts, in accordance with an embodiment of the present invention, AT1R inhibitor losartan can lower TNF, but cannot eliminate the difference between ACE7 (N-terminus KO) and wild type.

FIG. 13 depicts, in accordance with an embodiment of the present invention, an example of RXP 407.

FIG. 14 depicts, in accordance with an embodiment of the present invention, an example of RXPA 380.

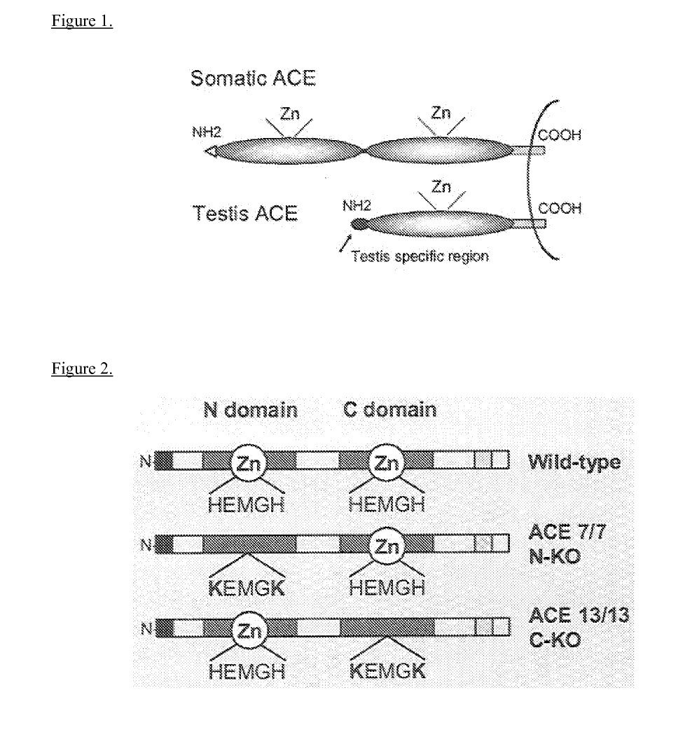

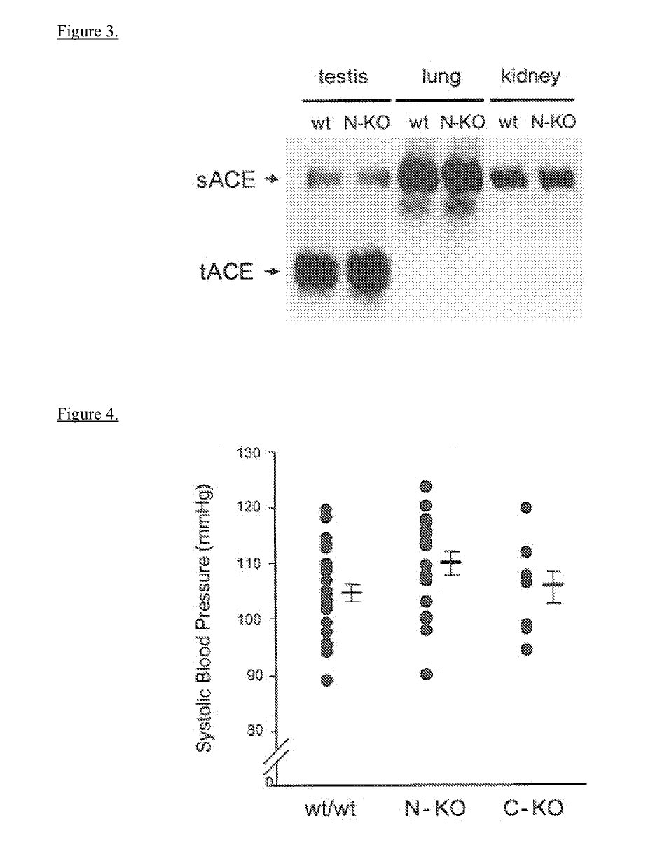

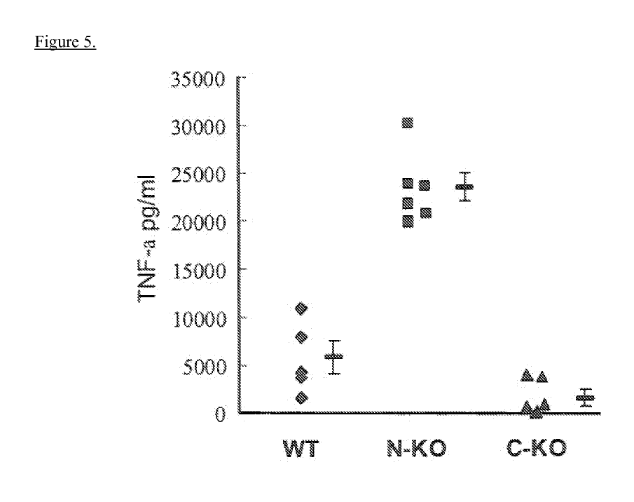

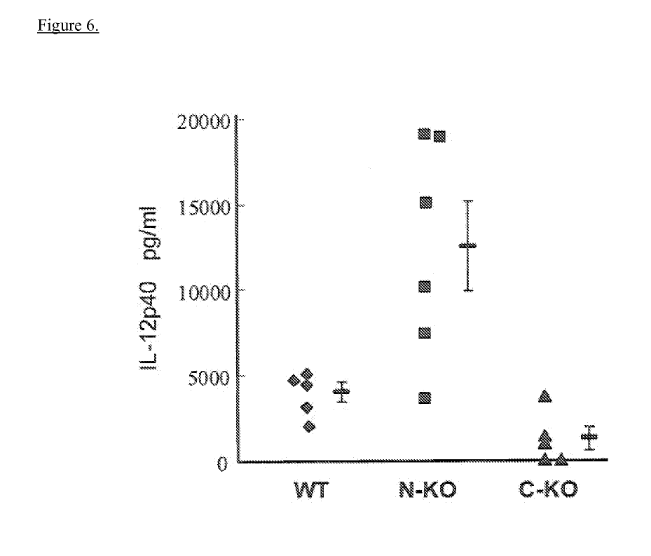

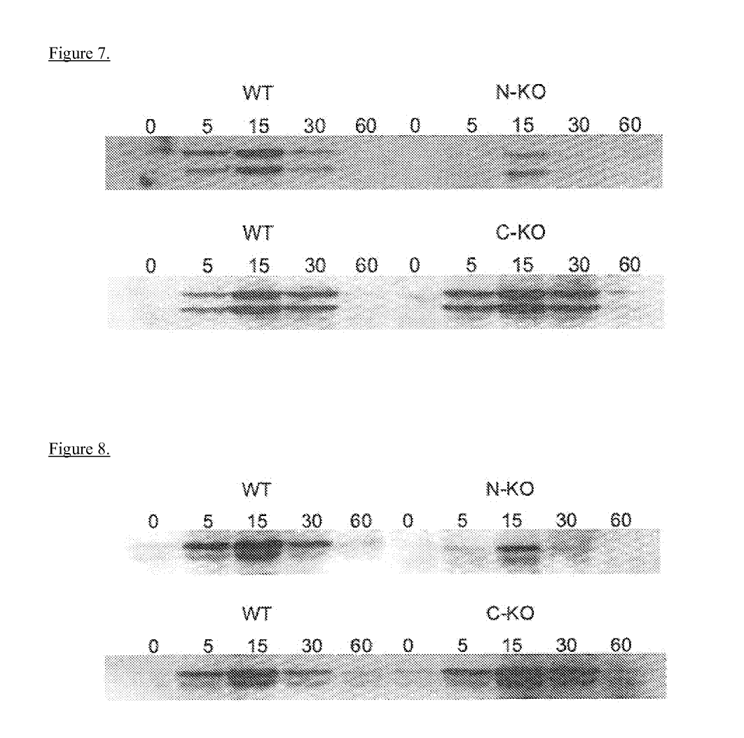

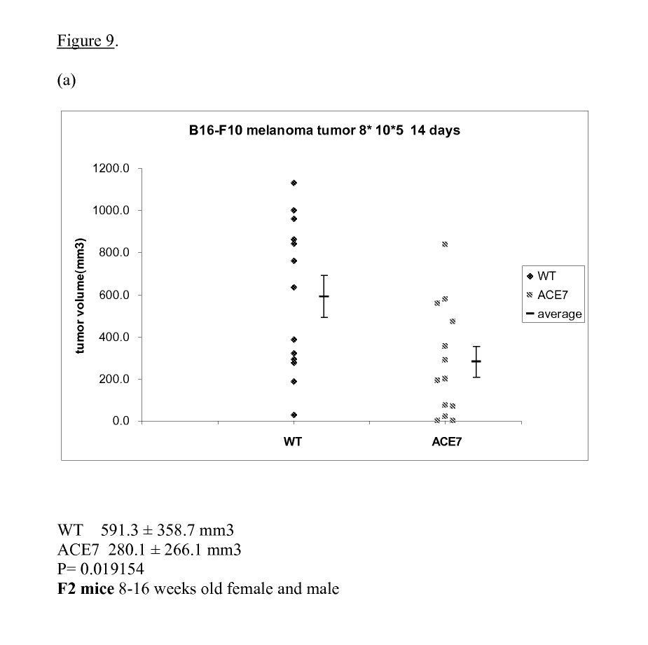

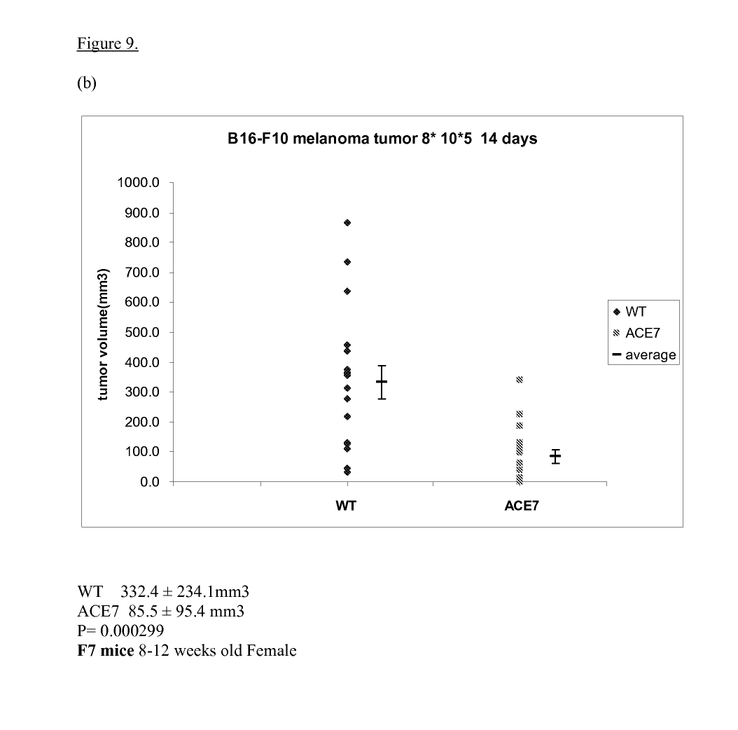

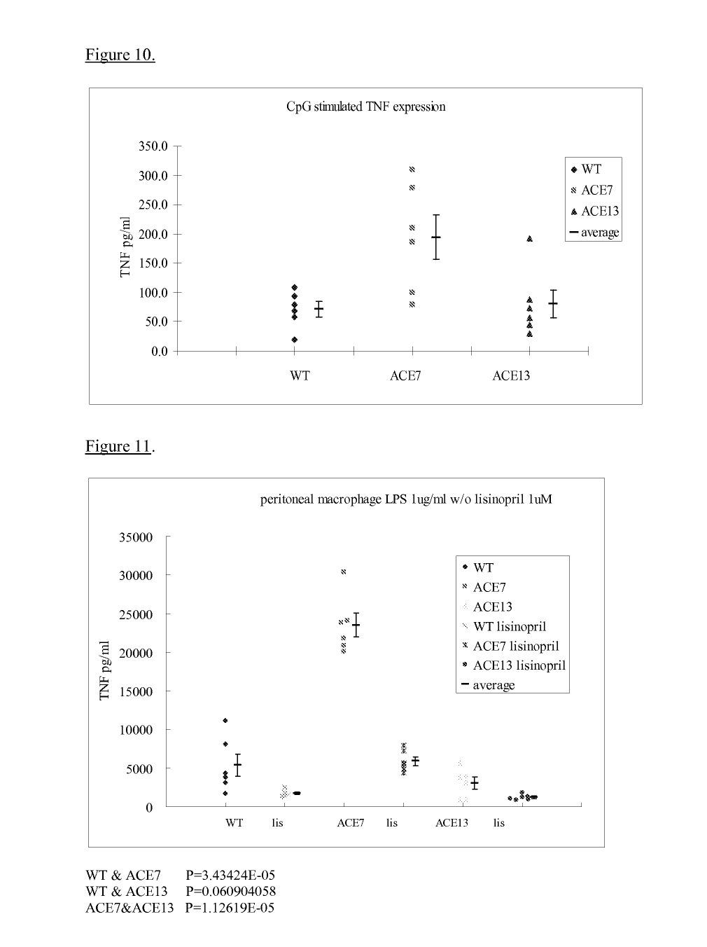

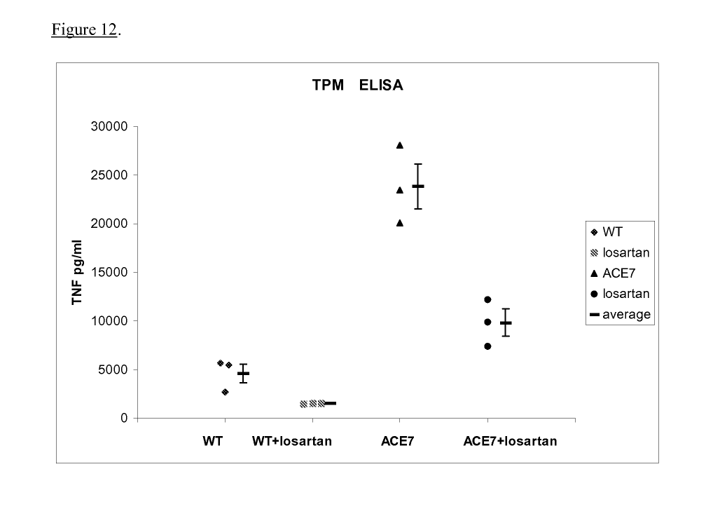

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

As used herein, “ACE 7/7 mice” are referred to as N-KO, and “ACE 13/13 mice” are referred to as “C-KO.” In other words, as disclosed herein, mice lines were utilized that lacked N-terminal ACE catalytic activity, referred to as N-KO, as well as mice lines that lacked C-terminal ACE catalytic activity, referred to as C-KO. “KO” is an abbreviation for “knock-out.”

As used herein, the term “ACE” refers to angiotensin converting enzyme.

As used herein, the term “AcSDKP” refers to Acetyl-SerAspLysPro (SEQ. ID. NO.: 1), a tertrapeptide that is cleaved from the ubiquitous protein thymosin-β4 by the enzyme prolyl oligopeptidase.

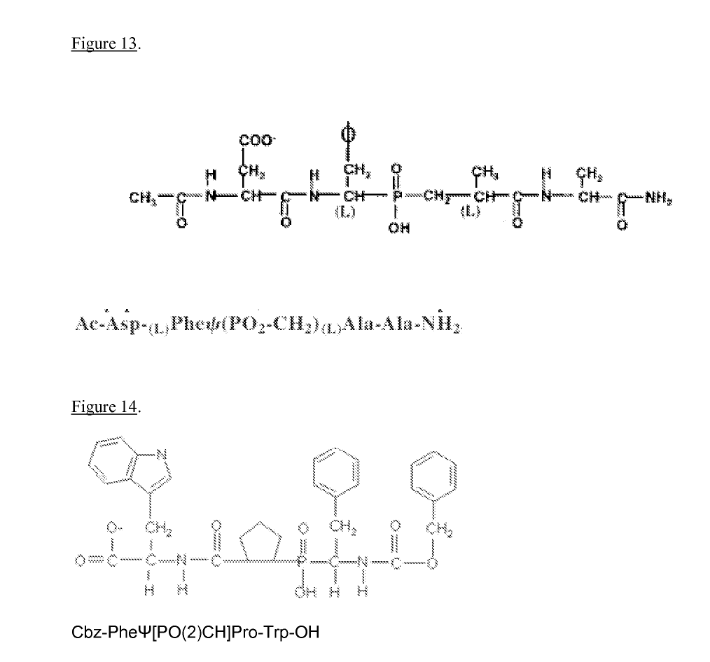

As used herein, the term “RXP 407” refers to a phosphinic peptide that may act as an inhibitor of angiotensin I converting enzyme able to differentiate between its two active sites. An example of RXP 407 is described in FIG. 13 herein, as well as in Dive, et al., Proc. Natl. Acad. Sci. Vol. 96, pp 4330-4335, April 1999, the contents of which are hereby incorporated by reference in its entirety.

As used herein, the term “RXPA 380” refers to a phosphinic peptide that may act as an inhibitor of angiotensin I converting enzyme able to differentiate between its two active sites, highly selective of the C-domain of ACE. An example of RXPA 380 is described in FIG. 14 herein, as well as in Acharya, et al., Nature Reviews 2: 891-902, 2003, the contents of which are hereby incorporated by reference in its entirety.

As disclosed herein, mice were created with point mutations that eliminated one or the other of the ACE domains (N-KO and C-KO). The result was that the inventors found that there was a large difference in the expression of cytokines, where the two ACE domains have different effects on cytokine expression. Today, ACE inhibitors are used to control blood pressure, heart disease and diabetes. However, all commercial ACE inhibitors inhibit both domains of ACE. Thus, results described herein allow the development of ACE site specific inhibitors (either the N- or C-terminus of ACE) in order to differentially effect cytokine expression and cell growth.

Various embodiments described herein pertain to the use of ACE domain specific inhibitors to control cytokine expression and/or disease, expanding previous concepts of selective inhibition of ACE.

In one embodiment, the present invention provides a method of developing and/or testing site specific ACE inhibitors by administering a site specific ACE inhibitor to a subject, followed by evaluating the resulting difference in ACE catalytic activity. In another embodiment, the administration of the site specific ACE inhibitor results in an increase and/or enhancement of cytokine expression. In another embodiment, the cytokine expression comprises TNF-α and IL-12. In another embodiment, the ACE inhibitor may specifically target the ACE N-terminus active domain. In another embodiment, the ACE inhibitor that specifically targets the ACE N-terminus domain comprises RXP 407, or a pharmaceutical equivalent, derivative, analog and/or salt thereof. In another embodiment, the ACE inhibitor may specifically target the ACE C-terminus active domain. In another embodiment, the ACE inhibitor that specifically targets the ACE C-terminus domain comprises RXPA 380, or a pharmaceutical equivalent, derivative, analog and/or salt thereof and/or ketomethylene inhibitors. In another embodiment, the ACE catalytic activity is evaluated by determining cytokine expression profiles. In another embodiment, the resulting difference in ACE catalytic activity may be evaluated by determining sensitivity to chloride concentration. In another embodiment, the subject is a rodent. In another embodiment, the subject is a human.

In one embodiment, the present invention provides a method of regulating and/or treating a condition in a subject, where the condition is associated with the presence of a peptide preferably cleaved by the N-terminus of ACE, comprising administering a therapeutically effective dosage of N-terminus site specific ACE inhibitor. In another embodiment, the condition is inflammation and/or immune response. In another embodiment, the peptide is AsSDKP. In another embodiment, the peptide is angiotensin I, bradykinin, substance P and/or β-endorphins. In another embodiment, the condition is cancer, melanoma, blood pressure, hematopoiesis, fertility, atherosclerosis, diabetes and/or kidney disease. In another embodiment, the N-terminus site specific ACE inhibitor comprises RXP 407, or a pharmaceutical equivalent, derivative, analog and/or salt thereof. In another embodiment, the subject is a rodent. In another embodiment, the subject is a human.

In another embodiment, the present invention provides a method of regulating and/or treating a condition in a subject, where the condition is associated with the presence of a peptide preferably cleaved by the C-terminus of ACE, comprising administering a therapeutically effective dosage of C-terminus site specific ACE inhibitor. In another embodiment, the condition is inflammation and/or immune response. In another embodiment, the peptide is angiotensin I, bradykinin, substance P and/or β-endorphins. In another embodiment, the ACE inhibitor that specifically targets the ACE C-terminus domain comprises RXPA 380, or a pharmaceutical equivalent, derivative, analog and/or salt thereof and/or ketomethylene inhibitors. In another embodiment, the condition is cancer, melanoma, blood pressure, hematopoiesis, fertility, atherosclerosis, diabetes and/or kidney disease. In another embodiment, the subject is a mouse. In another embodiment, the subject is a human.

As further disclosed herein, AcSDKP (and therefore ACE) may play an important role in regulating cardiac fibrosis in hypertension and heart failure. Acetyl-SerAspLysPro (AcSDKP) is a tetrapeptide that is cleaved from the ubiquitous protein thymosin-β4 by the enzyme prolyl oligopeptidase. AcSDKP was first described as a natural regulator of hematopoietic cell proliferation. The peptide is hydrolyzed and degraded by ACE, but while angiotensin I can be converted by both the C- and the N-terminal catalytic domains of ACE, AcSDKP is hydrolyzed almost exclusively by the N-terminal domain. Various reports indicate AcSDKP suppress fibroblast proliferation as one means of reducing cardiac fibrosis.

In one embodiment, the present invention provides a method of treating a condition regulated by AcSDKP by administering a therapeutically effective dosage of ACE inhibitor that specifically targets the ACE N-terminus domain. In another embodiment, the condition regulated by AcSDKP comprises hematopoietic cell proliferation. In another embodiment, the condition comprises suppression of fibroblast proliferation as a means of reducing cardiac fibrosis. In another embodiment, the N-terminus site specific ACE inhibitor comprises RXP 407, or a pharmaceutical equivalent, derivative, analog and/or salt thereof.

Further, as disclosed in Fuchs, et al., Nature Medicine, 11: 1140-1142, 2005, the contents of which are hereby incorporated by reference in its entirety, mice lacking C terminal activity lack testis ACE activity because testis ACE is a smaller protein only containing the C terminal. Thus, in one embodiment, the present invention provides a male contraceptive, where a specific inhibitor of the C terminal would cause poor reproduction for a male subject.

In various embodiments, the present invention provides pharmaceutical compositions including a pharmaceutically acceptable excipient along with a therapeutically effective amount of site specific ACE inhibitor. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective N-terminus or C-terminus site specific ACE inhibitor can be in the ranges recommended by the manufacturer where known therapeutic compounds are used, and also as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, such as biopsied malignant tumors, or the responses observed in the appropriate animal models, as previously described.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generally

Angiotensin converting enzyme (ACE) is composed of two homologous catalytic domains which are independently catalytic. Until now, most scientists knew that there were some differences between the two domains, but generally viewed the two domains as being fairly equivalent. For instance, there was not much data in vivo showing a significant difference of activity. As described herein, mice were created with point mutations that eliminated one or the other of the domains. In other words, using mice, in vivo data was produced. The inventors found that there was a big difference in the expression of cytokines. The finding that the two ACE domains have different effects on cytokine expression is very significant. Today, ACE inhibitors are used to control blood pressure, heart disease and diabetes. Sales of this class of medication are measured in the billions of dollars. However, all commercial ACE inhibitors inhibit both domains of ACE. Thus, results described herein allow the development of ACE site specific inhibitors (either the N- or C-terminus of ACE) in order to differentially effect cytokine expression and cell growth. Various embodiments described herein pertain to the use of ACE domain specific inhibitors to control cytokine expression and/or disease, expanding previous concepts of selective inhibition of ACE.

### Example 2

### Mouse Models with Inactivated N-Terminal or C-Terminal Catalytic Sites

To assess the physiologic significance of each of the two ACE catalytic domains, the inventors used gene targeting in ES cells to create two mouse models where the ACE gene was mutated to specifically inactivate either its N-terminal or C-terminal catalytic sites. Both the ACE N- and C-terminal domains contain the protein motif HEMGH (SEQ. ID. NO.: 2) which is necessary for zinc binding and catalytic activity; the introduction of two point mutations within ES cell DNA converts this motif to KEMGK (SEQ. ID. NO.: 3). This eliminates zinc binding and all catalytic activity of the modified domain. The first mouse line studied was termed ACE 7/7; this line lacks N-terminal ACE catalytic activity. The second mouse line is called ACE 13/13; here histidines within the C-terminal catalytic site were mutated to lysines. As used herein, ACE 7/7 mice are referred to as N-KO and ACE 13/13 mice as C-KO.

### Example 3

### Evaluation of Tissue Distribution of ACE

The tissue distribution of ACE was evaluated by enzyme activity assays and by Western blot analysis. A sample of the Western blot data from the N-KO mice is presented herein; data from C-KO mice were identical. These data, and a variety of other studies on both N-KO and C-KO mice established that the introduction of the point mutations has no effect on either the tissue pattern, the molecular size or the levels of ACE expression.

### Example 4

### Verification of Lack of N-KO and C-KO Catalytic Activity

The mutations used to create the N-KO and C-KO mice reflect extensive previous in vitro mutational analysis of ACE. These studies characterized mutations identical to those incorporated in N-KO and C-KO mice. However, to verify the biochemical phenotype, the inventors measured the catalytic activity of plasma using the synthetic ACE substrates AcSDAcKP (cleaved almost exclusively by the N-terminal) and Hip-His-Leu (HHL) (cleaved much more efficiently by the C-terminal). These studies substantiated the genetic changes. For example, N-KO mice cleaved AcSDAcKP with only 3±1% the efficiency of wild-type mice while cleavage of HHL was identical to wild-type. In contrast, C-KO mice hydrolyzed HHL with only 18±2% wild-type efficiency whereas AcSKAcKP was hydrolyzed at the same rate as wild-type. Another difference between the two ACE catalytic sites is the sensitivity to chloride concentration, and the inventors used that characteristic to prove that the residual 18% cleavage activity of HHL measured in C-KO plasma was due to the enzymatic activity of the N-terminal site. In summary, the enzymatic analysis substantiated published literature and verified that N-KO and C-KO mice lack catalytic activity in one of the two ACE domains.

### Example 5

### Systolic Blood Pressure

To study the physiology of the mice, systolic blood pressure was measured in wild-type (wt/wt) and the mutant mice. Systolic blood pressure was obtained in trained mice by repetitive tail cuff measurements over 4 days. Individual data points and group means are shown herein. Statistical analysis showed no meaningful difference between these groups of mice. Thus, under steady state conditions, there is no difference in blood pressure between N-KO, C-KO and wild-type mice. Further, neither N-KO nor C-KO showed renal pathology or any defect in renal concentrating ability.

### Example 6

### Differences in Behavior Between Macrophages Derived from N-KO and C-KO Mice

The inventors demostrate that there are significant differences in behavior between macrophages derived from N-KO and C-KO mice, offering significant functional differences between the two catalytic domains of ACE. Thus, the inventors demonstrate novel approaches for regulating the inflammatory response and towards inflammatory processes in general.

To obtain large numbers of macrophages, N-KO, C-KO and WT mice were injected intraperitoneally with thioglycolate. After four days, cells were collected, washed and counted. Aliquots of 1×10⁶ cells were adhered in 24 well plates. After 2 hrs, non-adherent cells were removed and the plates were visually evaluated to verify that wells contained nearly identical numbers of adherent cells. Media containing 1 ug/ml of LPS (a model activator of macrophages) was then added. After 24 hrs, supernatants were collected and the concentrations of TNF-α and IL-12p40 (one of the 2 proteins comprising IL-12) were determined by ELISA (eBioscience). However, there was a marked difference in cytokine expression profile; macrophages derived from N-KO mice producing far more TNF- a and IL-12p40 than either C-KO or WT mice (p values for TNF-α: N-KO vs. C-KO or WT, p<0.001; p values for IL12p40: N-KO vs. C-KO or WT, p<0.02). Further, there was a clear trend of macrophages from C-KO mice producing less cytokines than wild-type cells (TNF- a: C-KO vs. WT, p<0.07; IL12p40: C-KO vs. WT, p<0.02). Thus, in the absence of ACE N-terminal catalytic activity, TNF-α and IL-12 cytokine expression was increased. In the absence of C-terminal catalytic activity, there is a strong reduction of production of these cytokines. It should be noted that enhanced production of TNF-α and IL-12 in response to LPS is a typical feature of an M1 macrophage response, while little or no production of these cytokines is characteristic of M2 macrophages. Thus, at the very least, these data indicate a striking difference in LPS stimulated cytokine profiles between macrophages derived from N-KO, C-KO and WT mice.

### Example 7

### Physiologic Differences Between Catalytic Activities

The data might pertain to something about the peptide environment in the N-KO mice promoting macrophage development toward the M1 phenotype, where the reduced fibrosis observed 2 weeks after bleomycin injury in N-KO mice would be due to a more effective acute response with more efficient resolution of the acute injury and a relative suppression of the M2 (pro-fibrotic) macrophage population. Alternatively, the environment in the N-KO mice may generally suppress macrophage activation, but that when these cells are removed from the mouse and placed into tissue culture they rebound and show an exaggerated cytokine response, where then the reduced bleomycin injury observed in vivo in the N-KO mice may be a function of a generalized suppression of the inflammatory response. Regardless, the asymmetry in macrophage cytokine profiles in N-KO and C-KO mice and the in vivo difference in response to intratracheal bleomycin is strong evidence of significant physiologic differences between the catalytic activities of the N- and C-terminal catalytic domains of ACE. The results extend to injury beyond just bleomycin.

### Example 8

### N-KO mice have Reduction of p-JNK and p-Erk1/2 Levels

The inventors demonstrate differences in cytokine expression by macrophages derived from N-KO and C-KO mice. To further expand on this, the inventors studied the intracellular signaling pathways known to regulate cytokine expression in response to LPS. Thioglycolate elicited peritoneal macrophages were isolated from N-KO, C-KO and littermate WT mice. The macrophages were washed and adhered to 100 mm plastic plates. After 2 hrs, they were washed again to remove non-adherent cells and 1 :g/ml LPS in RPMI 1640 with 5% FCS was added. Individual plates were harvested 0, 5, 15, 30 and 60 min after LPS stimulation. To harvest cells, they were washed with iced PBS and then lysed with lysis buffer containing 1% NP-40, and inhibitors of serine, aspartyl, and cysteine proteases. Protein concentration was determined and then, for each time point, 100 :g of protein were analyzed by Western blot analysis. The blots were probed sequentially with anti-phosphorylated INK (p-JNK), and anti-phosphorylated Erk1/2 (p-Erk1/2)(Cell Signal Technology). Both p-JNK and p-Erk1/2 are the catalytically active forms of these MAP kinases. Finally, the blot was probed with anti-total Erk1/2 to verify equal loading. These data show a marked asymmetry of response of macrophages from the N-KO and C-KO mice. Macrophages from wild-type mice show the expected increase of p-JNK and p-Erk at 5 min with maximal levels at 15 min The macrophages from N-KO mice have a marked reduction of the p-JNK and p-Erk1/2 levels (total Erk probing showed equal loading). In contrast, macrophages from C-KO animals showed phosphorylation levels that are at least equivalent to those of wild-type cells and probably even increased. The experiments interestingly showed changes in N-KO and C-KO cells that are opposite in direction from each other. This is similar to the inventors' data for cytokine expression which also showed opposite changes in the N-KO and C-KO cells. Finally, the data interestingly demonstrates that N-KO cells, which are suppressed in their .INK and Erk1/2 response when this is measured 2 hrs after cell removal from the mice, have increased TNF-α expression in a 24 hr cytokine assay. Typically, LPS stimulation of TNF- a is associated with increased activation of MAP kinases. Importantly, again, the data shows a substantial difference in the ACE N- and C-terminal domain effect, this time as measured in a very standard biochemical assay.

### Example 9

### Site Specific ACE Inhibitors

There are presently some examples of specialty peptides—used up to this point only in research—which specifically inhibit either N- or C-terminal ACE activity, but not both. For example, RXP 407, a peptide in which a phosphinic acid bond is used in place of a peptide bond, has a dissociation constant three orders of magnitude lower for the ACE N-domain than for the C-terminal domain. This compound is reported as stable in vivo and when used in a mouse increases the plasma level of Ac-SDKP as much as 6-fold. When combined with Ac-SDKP infusion, a 16-fold elevation of plasma Ac-SDKP was obtained. Another phosphinic peptide, RXPA 380 is reported as being a C-terminal specific inhibitor, with a dissociation constant more than 3 orders of magnitude lower for the ACE C-domain than the N-domain. Again, this compound appears stable and effective in mice. Finally, a different chemical class of C-terminal specific ACE inhibitors, termed ketomethylene inhibitors, has been previously prepared and tested. Again, there is a 3-order of magnitude difference in dissociation constant, with very little effect on the N-terminal of ACE.

### Example 10

### Biochemical Properties of AcSDKP

Acetyl-SerAspLysPro (AcSDKP) is a tetrapeptide that is cleaved from the ubiquitous protein thymosin-β by the enzyme prolyl oligopeptidase. AcSDKP was first described as a natural regulator of hematopoietic cell proliferation. The peptide is hydrolyzed and degraded by ACE, but while angiotensin I can be converted by both the C- and the N-terminal catalytic domains of ACE, AcSDKP is hydrolyzed almost exclusively by the N-terminal domain. A 7-fold increase in the plasma concentration of AcSDKP after the acute administration of an ACE inhibitor to normal volunteers shows the important in vivo role of ACE in the regulation of this peptide.

Several papers suggest that AcSDKP (and therefore ACE) may play an important role in regulating cardiac fibrosis in hypertension and heart failure. Peng et al. (Hypertension 42: 1164-1170, 2003) reported that in 2-kidney, 1-clip hypertensive rats, administration of AcSDKP by osmotic mini-pump increased plasma AcSDKP to a similar degree as observed in patients treated with ACE inhibitors. While this had no effect on blood pressure, AcSDKP prevented the development of fibrosis in the heart. Further work by Carretero and colleagues (Am J Physiol Heart Circ Physiol. 294: 1226-32, 2008) established that AcSDKP reduced inflammation by effects on macrophages, including reducing macrophage production of TNF. Another experiment showed that, in aldosterone-salt hypertension, administration of AcSDKP prevented increased collagen deposition and cell proliferation in the heart and kidney. AcSDKP also reduced aortic fibrosis secondary to angiotensin II-induced hypertension. Furthermore, AcSDKP inhibits the growth of cardiac fibroblasts and inhibits transforming growth factor (TGF)-β1 stimulated phosphorylation of Smad2. Taken together, these reports suggest that AcSDKP may suppress fibroblast proliferation as one means of reducing cardiac fibrosis.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

1. Corvol P, Williams T A, Soubrier F. Peptidyl dipeptidase A: angiotensin I-converting enzyme. Methods Enzymol. 248: 283-305, 1995
2. Metzger R, Bohle R M, Chumachenko P, Danilov S M, Franke F E. CD143 in the development of atherosclerosis. Atherosclerosis. 150: 21-31, 2000
3. Metzger R, Bohle R M, Pauls K, Eichner G, Alhenc-Gelas F, Danilov S M, Franke F E. Angiotensin-converting enzyme in non-neoplastic kidney diseases. Kidney Int. 56: 1442-1454, 1999
4. Leehey D J, Singh A K, Alavi N, Singh R. Role of angiotensin II in diabetic nephropathy. Kidney Int Suppl. 77: S93-S98, 2000
5. Nguyen L, Ward W F, Ts'ao C H, Molteni A. Captopril inhibits proliferation of human lung fibroblasts in culture: a potential antifibrotic mechanism. Proc Soc Exp Biol Med. 205: 80-84, 1994
6. Soubrier F, Alhenc-Gelas F, Hubert C, Allegrini J, John M, Tregear G, Corvol P. Two putative active centers in human angiotensin I-converting enzyme revealed by molecular cloning. Proc. Natl. Acad. Sci. USA 85:9386-90, 1988
7. Bernstein K E, Martin B M, Edwards A S, Bernstein E A. Mouse angiotensin I-converting enzyme is a protein composed of two homologous domains. J Biol Chem 264:11945-11951, 1989.
8. Wei L, Alhenc-Gelas F, Corvol P, Clauser E. The two homologous domains of human angiotensin I-converting enzyme are both catalytically active. J Biol Chem. 266: 9002-9008, 1991
9. Jaspard E, Wei L, Alhenc-Gelas F. Differences in the properties and enzymatic specificities of the two active sites of angiotensin I-converting enzyme (kininase II). Studies with bradykinin and other natural peptides. J Biol Chem. 268: 9496-9503, 1993
10. Wei L, Clauser E, Alhenc-Gelas F, Corvol P. The two homologous domains of human angiotensin I-converting enzyme interact differently with competitive inhibitors. J Biol Chem. 267: 13398-13405, 1992
11. Rousseau A, Michaud A, Chauvet M T, Lenfant M, Corvol P. The hemoregulatory peptide N-acetyl-Ser-Asp-Lys-Pro is a natural and specific substrate of the N-terminal active site of human angiotensin-converting enzyme. J Biol Chem. 270: 3656-3661, 1995
12. Fuchs S, Frenzel K, Xiao H D, Adams J W, Zhao H, Keshelava G, Teng L, Bernstein K E. Newly Recognized Physiologic and Pathophysiologic Actions of the Angiotensin-converting Enzyme. Curr Hypertens Rep. 6: 124-128, 2004
13. Weber K T. Fibrosis, a common pathway to organ failure: angiotensin II and tissue repair. Semin Nephrol. 17: 467-491, 1997
14. Tikellis C, Wookey P J, Candido R, Andrikopoulos S, Thomas M C, Cooper M E. Improved islet morphology after blockade of the renin-angiotensin system in the ZDF rat. Diabetes. 53: 989-997, 2004
15. Kawaguchi Y, Takagi K, Hara M, Fukasawa C, Sugiura T, Nishimagi E, Harigai M, Kamatani N. Angiotensin II in the lesional skin of systemic sclerosis patients contributes to tissue fibrosis via angiotensin II type 1 receptors. Arthritis Rheum. 50: 216-226, 2004
16. Otsuka M, Takahashi H, Shiratori M, Chiba H, Abe S. Reduction of bleomycin induced lung fibrosis by candesartan cilexetil, an angiotensin II type 1 receptor antagonist. Thorax. 59: 31-38, 2004
17. Tuncer I, Ozbek H, Ugras S, Bayram I. Anti-fibrogenic effects of captopril and candesartan cilexetil on the hepatic fibrosis development in rat. The effect of AT1-R blocker on the hepatic fibrosis. Exp Toxicol Pathol. 55: 159-166, 2003
18. Tokuda K, Kai H, Kuwahara F, Yasukawa H, Tahara N, Kudo H, Takemiya K, Koga M, Yamamoto T, Imaizumi T. Pressure-independent effects of angiotensin II on hypertensive myocardial fibrosis. Hypertension. 43: 499-503, 2004
19.Challah M, Nicoletti A, Arnal J F, Philippe M, Laboulandine I, Allegrini J, Alhenc-Gelas F, Danilov S, Michel J B. Cardiac angiotensin converting enzyme overproduction indicates interstitial activation in renovascular hypertension. Cardiovasc Res. 30:231-9, 1995
20. Vio C P, Jeanneret V A. Local induction of angiotensin-converting enzyme in the kidney as a mechanism of progressive renal diseases. Kidney Int Suppl. S57-S63, 2003
21. Wynn T A. Cellular and molecular mechanisms of fibrosis. J Pathol. 214:199-210, 2008
22. Booz G W, Baker K M., Molecular signalling mechanisms controlling growth and function of cardiac fibroblasts. Cardiovasc Res. 30: 537-543, 1995
23. Murasawa S, Matsubara H, Mori Y, Masaki H, Tsutsumi Y, Shibasaki Y, Kitabayashi I, Tanaka Y, Fujiyama S, Koyama Y, Fujiyama A, Iba S, Iwasaka T. Angiotensin II initiates tyrosine kinase Pyk2-dependent signalings leading to activation of Rac1-mediated c-Jun NH2-terminal kinase. J Biol Chem. 275: 26856-26863, 2000
24. Brilla C G, Funck R C, Rupp H. Lisinopril-mediated regression of myocardial fibrosis in patients with hypertensive heart disease. Circulation. 102: 1388-1393, 2000
25. Brilla C G, Janicki J S, Weber K T. Impaired diastolic function and coronary reserve in genetic hypertension. Role of interstitial fibrosis and medial thickening of intramyocardial coronary arteries. Circ Res. 69: 107-115, 1991
26. Gagnon C, Legault F, Geraides P, Tanguay J F, Lambert C. Diverse effects of Ace inhibitors and angiotensin II receptor antagonists on prevention of cardiac hypertrophy and collagen distribution in spontaneously hypertensive rats. Int J Cardiol. 97: 373-381, 2004
27. Gross O, Schulze-Lohoff E, Koepke M L, Beirowski B, Addicks K, Bloch W, Smyth N, Weber M. Antifibrotic, nephroprotective potential of ACE inhibitor vs AT1 antagonist in a murine model of renal fibrosis. Nephrol Dial Transplant. 19: 1716-1723, 2004
28. Schanstra J P, Duchene J, Desmond L, Neau E, Calise D, Estaque S, Girolami J P, Bascands J L. The protective effect of angiotensin converting enzyme inhibition in experimental renal fibrosis in mice is not mediated by bradykinin B2 receptor activation. Thromb Haemost. 89: 735-740, 2003
29. Pradelles P, Frobert Y, Créminon C, Ivonine H, Frindel E. Distribution of a negative regulator of haematopoietic stem cell proliferation (AcSDKP) and thymosin beta 4 in mouse tissues. FEBS Lett. 289: 171-175, 1991
30. Azizi M, Rousseau A, Ezan E, Guyene T T, Michelet S, Grognet J M, Lenfant M, Corvol P, Ménard J. Acute angiotensin-converting enzyme inhibition increases the plasma level of the natural stem cell regulator N-acetyl-seryl-aspartyl-lysyl-proline. J Clin Invest. 97: 839-844, 1996
31. Azizi M, Ezan E, Nicolet L, Grognet J M, Ménard J. High plasma level of N-acetyl-seryl-aspartyl-lysyl-proline: a new marker of chronic angiotensin-converting enzyme inhibition. Hypertension. 30: 1015-1019, 1997
32. Peng H, Carretero O A, Brigstock D R, Oja-Tebbe N, Rhaleb N E. Ac-SDKP reverses cardiac fibrosis in rats with renovascular hypertension. Hypertension. 42: 1164-1170, 2003
33. Yang F, Yang XP, Liu YH, Xu J, Cingolani O, Rhaleb N E, Carretero O A. Ac-SDKP reverses inflammation and fibrosis in rats with heart failure after myocardial infarction. Hypertension. 43: 229-236, 2004
34. Sharma U, Rhaleb N E, Pokharel S, Harding P, Rasoul S, Peng H, Carretero O A. Novel anti-inflammatory mechanisms of N-Acetyl-Ser-Asp-Lys-Pro in hypertension-induced target organ damage. Am J Physiol Heart Circ Physiol. 294:H1226-32, 2008.
35. Peng H, Carretero O A, Raij L, Yang F, Kapke A, Rhaleb N E. Antifibrotic effects of N-acetyl-seryl-aspartyl-Lysyl-proline on the heart and kidney in aldosterone-salt hypertensive rats. Hypertension. 37: 794-800, 2001
36. Lin C X, Rhaleb N E, Yang X P, Liao T D, D'Ambrosio M A, Carretero O A. Prevention of aortic fibrosis by N-acetyl-seryl-aspartyl-lysyl-proline in angiotensin II-induced hypertension. Am J Physiol Heart Circ Physiol. 295:H1253-H1261, 2008
37. Pokharel S, Rasoul S, Roks A J, van Leeuwen R E, van Luyn M J, Deelman L E, Smits J F, Carretero O, van Gilst W H, Pinto YM. N-acetyl-Ser-Asp-Lys-Pro inhibits phosphorylation of Smad2 in cardiac fibroblasts. Hypertension. 40: 155-161, 2002
38. Dive V, Cotton J, Yiotakis A, Michaud A, Vassiliou S, Jiracek J, Vazeux G, Chauvet MT, Cuniasse P, Corvol P. RXP 407, a phosphinic peptide, is a potent inhibitor of angiotensin I converting enzyme able to differentiate between its two active sites. Proc Natl Acad Sci U S A. 96:4330-5, 1999
39. Junot C, Gonzales M F, Ezan E, Cotton J, Vazeux G, Michaud A, Azizi M, Vassiliou S, Yiotakis A, Corvol P, Dive V. RXP 407, a selective inhibitor of the N-domain of angiotensin I-converting enzyme, blocks in vivo the degradation of hemoregulatory peptide acetyl-Ser-Asp-Lys-Pro with no effect on angiotensin I hydrolysis. J Pharmacol Exp Ther. 297:606-11, 2001
40. Georgiadis D, Beau F, Czarny B, Cotton J, Yiotakis A, Dive V. Roles of the two active sites of somatic angiotensin-converting enzyme in the cleavage of angiotensin I and bradykinin: insights from selective inhibitors. Circ Res. 25;93:148-54, 2003
41. Watermeyer J M, Kroger W L, O'Neill H G, Sewell B T, Sturrock E D. Probing the basis of domain-dependent inhibition using novel ketone inhibitors of Angiotensin-converting enzyme. Biochemistry. 47:5942-50, 2008
42. Fuchs S, Xiao H D, Adams J W, Frenzel K, Keshelava G, Capecchi M R, Corvol P, Bernstein K E. Role of the N-terminal catalytic domain of ACE investigated by targeted inactivation in mice. J. Biol. Chem., 279: 15946-15953, 2004
43. Fuchs S, Xiao H D, Michaud A, Campbell D J, Hubert C, Capecchi M R, Corvol P, Bernstein K E. The ACE C-terminal catalytic domain is the main site of angiotensin I cleavage in vivo. Hypertension, 51:267-74, 2008
44. Gasse P, Mary C, Guenon I, Noulin N, Charron S, Schnyder-Candrian S, Schnyder B, Akira S, Quesniaux V F, Lagente V, Ryffel B, Couillin I. IL-1R1/MyD88 signaling and the inflammasome are essential in pulmonary inflammation and fibrosis in mice. J Clin Invest. 117:3786-99, 2007

1. A method of regulating an inflammatory condition in a subject, comprising:
obtaining a sample from the subject;
assaying the sample to determine the presence of a cytokine expression profile associated with an angiotensin converting enzyme (ACE) site specific catalytic domain; and
regulating the inflammatory condition by administering to the subject a therapeutically effective amount of an inhibitor specific to the ACE site specific catalytic domain.

2. The method of claim 1, wherein the ACE site specific catalytic domain is an ACE N-terminal site domain.

3. The method of claim 1, wherein the ACE site specific catalytic domain is an ACE C-terminal site domain.

4. The method of claim 1, wherein the cytokine expression profile comprises an TNF-α, IL-12, and/or IL-6 overexpression.

5. The method of claim 4, wherein the TNF-α, IL-12, and/or IL-6 overexpression is associated with inactivation of an ACE N-terminal site domain.

6. The method of claim 1, wherein the cytokine expression profile is associated with a MAP kinase expression profile.

7. The method of claim 6, wherein the MAP kinase expression profile comprises phosphorylated JNK and/or Erk levels.

8. The method of claim 1, wherein the cytokine expression profile is produced by interferon-γ (IFN-γ) and/or lipopolysacharide (LPS) stimulation.

9. The method of claim 1, wherein the inhibitor specific to the ACE site specific catalytic domain comprises a compound the formula: 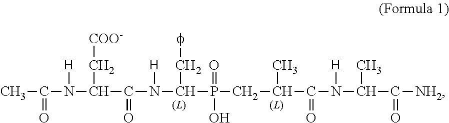
or a pharmaceutical equivalent, derivative, analog and/or salt thereof.

10. The method of claim 1, wherein the inhibitor specific to the ACE site specific catalytic domain comprises a compound the formula: 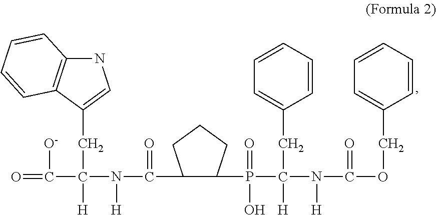
or a pharmaceutical equivalent, derivative, analog and/or salt thereof and/or ketomethylene inhibitors.

11. The method of claim 1, wherein the subject is a human.

12. The method of claim 1, wherein the subject is a rodent.

13. The method of claim 1, wherein the condition is regulated by AcSDKP.

14. The method of claim 1, wherein the condition is regulated by angiotensin I, bradykinin, substance P and/or β-endorphins.

15. A method of treating a disease and/or condition associated with angiotensin converting enzyme (ACE) in a subject, comprising:
providing a site specific ACE inhibitor; and
treating the condition by administering a therapeutically effective dosage of the site specific ACE inhibitor to the subject.

16. The method of claim 15, wherein the subject is a human.

17. The method of claim 15, wherein the subject is a rodent.

18. The method of claim 15, wherein the disease and/or condition associated with ACE is cancer.

19. The method of claim 15, wherein the disease and/or condition associated with ACE is melanoma.

20. The method of claim 15, wherein the disease and/or condition associated with ACE is blood pressure, hematopoiesis, fertility, atherosclerosis, diabetes and/or kidney disease.

21. The method of claim 15, wherein the site specific ACE inhibitor comprises an ACE N-terminal domain inhibitor.

22. The method of claim 15, wherein the site specific ACE inhibitor comprises an ACE C-terminal domain inhibitor.

23. A method of treating cancer in a subject, comprising:
providing a composition comprising an agent that substantially inactivates the N-terminal catalytic domain of angiotensin-converting enzyme (ACE); and
administering a therapeutically effective amount of the composition to the subject.

24. The method of claim 23, wherein the subject is a rodent.

25. The method of claim 23, wherein the subject is a human.

26. The method of claim 23, wherein the cancer is melanoma.

27. A method of developing and/or screening for an angiotensin converting enzyme (ACE) inhibitor, comprising:
determining the presence of an ACE domain that has an effective catalytic action; and
developing an ACE inhibitor that specifically catalyses the ACE domain.
